(19) United States
(12) Patent Application Publication
(10) Pub. No.: US 2011/0175245 A1
(43) Pub. Date: Jul. 21, 2011

(54) ENCAPSULATION

(75) Inventors: Nicola Tirelli, Manchester (GB); Giona Kilcher, Manchester (GB); Federica Ciamponi, Piombino (IT)

(73) Assignee: THE UNIVERSITY OF MANCHESTER, Manchester (GB)

(21) Appl. No.: 12/734,281

(22) PCT Filed: Oct. 27, 2008
(86) PCT No.: PCT/GB2008/003615

§ 371 Date: Feb. 2, 2011

(30) Foreign Application Priority Data
Oct. 25, 2007 (GB) 0721005.7

(51) Int. Cl. A61K 9/50; A61J 3/07

(52) U.S. Cl. 264/4

(57) ABSTRACT

A method of encapsulation comprises the steps of: (a) treating microbial microcapsules with a material to be encapsulated in a substantially anhydrous liquid medium containing a polar aprotic solvent having a dielectric constant greater than 35 under conditions to permit encapsulation of the material, and (b) subjecting the product of step (a) to a substantially aqueous liquid. The preferred solvent is dimethyl sulfoxide (DMSO). The method of the invention is useful for encapsulating hydrophobic and/or high molecular weight materials in yeast.

The present invention relates to encapsulation and more particularly to the encapsulation of materials into microbial microcapsules, e.g. algae, bacteria or fungi (most preferably a yeast). The method of the invention is suitable particularly, but not necessarily exclusively for the encapsulation of hydrophobic/lipophilic materials into micobrial microcapsules.

Products comprising a microbial microcapsule containing an encapsulated (exogenous) material have been known for many years. A particular feature of such products is that the microbial microcapsule acts as a carrier to “deliver” the encapsulated material, the cell wall or membrane of the microcapsule serving as a barrier to protect and/or preserve the encapsulated material until such time as it is required for use. At this time, the microcapsule may be degraded or otherwise destroyed by the conditions under which the product is used so as to release the previously encapsulated material.

In such products, the microcapsule may be an alga, bacteria or fungus (e.g. yeast) and the encapsulated material is frequently lipophilic or hydrophobic. Depending on the nature of the microcapsule and the encapsulated material, the product may be for food, agrochemical, pharmaceutical or industrial use. Thus the encapsulated material can be a flavouring, essential oil, pharmaceutical, nutraceutical, or agrochemical. In the case of relatively volatile flavourings, for example, the microcapsule serves to prevent loss of the flavouring by evaporation.

By way of more specific illustration, one example of such a product comprises yeast cells (as the microcapsule) incorporating encapsulated garlic oil retained in the cells by the membrane thereof. Such a product may be used for making a garlic-flavoured bread by mixing the product into the dough from which the bread is baked with the result that, during proving, the garlic oil components are prevented from permeating the dough and inhibiting the respiration of the yeast or chemically inhibiting structure development. The volatile flavour components are retained during the baking process as the yeast cells capsules remain intact and release the garlic flavouring directly into the mouth during eating. A particular advantage of this technique is that the yeast acts as a carrier to release the garlic oil substantially only during consumption. This is in contrast to the case of adding “free” garlic oil to the dough since this can result in loss of structure and reduced bread volume and the loss of flavouring to the atmosphere, e.g. during mixing of the dough and transfer to an oven in which the dough is baked.

Various examples of techniques employed for encapsulating materials in microbial microcapsules are summarised below.

EP-A-0 085 805 (Dunlop Ltd) discloses a method of producing a microcapsule incorporating an encapsulated product. In this method, the lipid content of a grown microbe is extended by treating the grown microbe with an organic, lipid-extending substance which is taken up by the microbe and retained passively therein. The lipid extending substance may be selected, for example, from aliphatic alcohols, esters, aromatic hydrocarbons and hydrogenated aromatic hydrocarbons. The lipid-extending substance may itself be the product to be encapsulated. Additionally however the microbe may be treated with a material which is soluble or micro-dispersable in the organic lipid-extending substance so that material is taken into the extended lipid of the microbe and retained passively therein. This latter material may, for example, be a lecuo dye to provide a product that is useful in the production of carbonless copying paper.

GB-A-2 162 147 discloses a method of making an encapsulated product by contacting a grown microbe having less than 10% by weight lipid content with an organic liquid which is capable of entering the microbe by diffusion through the microbial cell wall without rupture thereof. Examples of suitable organic liquids are stated to be alcohols, esters and some liquid hydrocarbons. The organic liquid may itself be the material to be encapsulated. Alternatively the organic liquid may be employed as a solvent or dispersant for the material to be encapsulated. Examples of encapsulatable materials (apart from the organic liquids themselves) are given as dyes, various types of pesticides, e.g. insecticides, herbicides etc), pheromones, odiferous materials (e.g. perfumes), flavourants and pharmaceuticals.

EP-A-0 242 135 discloses production of an encapsulated material by treating a grown intact microbe (such as a fungus, bacterium or alga) by contiguous contact with an encapsulatable material in liquid form. The microbe has a microbial lipid content of significantly less than 40% by weight and the encapsulatable material is capable of diffusing into the microbial cell without causing total lysation thereof. The method is effected in the absence of an organic lipid-extending substance of the type employed in EP-A-0 085 805 (see above) and also in the absence of a plasmolyser.

One feature of the prior processes is that the processes are restricted to small lipophilic or hydrophilic molecules and principally molecules with partition coefficients below Log P 4.0 and molecular weights below 600 Da.

It is an object of the present invention to obviate or mitigate the aforementioned disadvantages.

According to the present invention there is provided a method of encapsulation comprising the steps of

(a) treating microbial microcapsules with a material to be encapsulated in a substantially anhydrous liquid medium containing a polar aprotic solvent having a dielectric constant greater than 35 under conditions to permit encapsulation of the material, and

(b) subjecting the product of step (a) to a substantially aqueous liquid.

By “substantially anhydrous” we mean that the liquid medium employed in step (a) contains less than 50% by weight of water based on the weight of the liquid medium, including any water taken up from the microbial microcapsules. More preferably, the medium contains less than 25%, even more preferably less than 10% and ideally less than 5% by weight of water on the same basis. In the most preferred embodiments of the invention, the microbial microcapsules to be treated have been separated (e.g. by centrifugation) from any free aqueous medium with which they may have been associated or dried (e.g. by spray drying) and the liquid medium is the polar aprotic solvent in anhydrous form.

By “substantially aqueous” liquid (as employed for step (b)) we mean a liquid which comprises at least 50% by weight of water, more preferably at least 75%, even more preferably at least 90%, and ideally at least 95% by weight water. We do not however preclude the possibility that the treated microbial microcapsules obtained from step (a) are initially subjected to an environment which comprises a major proportion of the polar aprotic solvent and a minor proportion of water so as to prevent “shock” to the microbial microcapsules before they are subjected to the substantially aqueous liquid.

In contrast to the prior art discussed above which teaches generally the use of relatively non-polar solvents in methods of encapsulating materials in microbial microcapsules we have found that polar aprotic solvents with relatively high dielectric constants (i.e. at least 35) provide for solvent mediated encapsulation of materials into microbial microcapsules. We believe that under the substantially anhydrous conditions of the method, the polar aprotic solvent serves to swell the molecular network of the cell wall or membrane of the microbial microcapsule to allow permeation of the material to be encapsulated. Subsequently the microbial cells that have been treated with the polar, aprotic solvent are subjected to a substantially aqueous environment which we have found reverses the permeability change to allow for the encapsulated materials to remain incorporated.

The method of the invention is particularly effective, although not only for the encapsulation of materials which are substantially hydrophobic (i.e. water insoluble), and/or which have a log P value greater than 2 and/or which have a molecular weight greater than 400 Da.

More specifically the material to be encapsulated may have a log P value of greater than 3 or greater than 4.

Alternatively or additionally the material to be encapsulated may have a molecular weight of greater than 700 Da, e.g. greater than 1000 Da or greater than 2000 Da, e.g. up to 5000 Da. Thus the method may be used for the encapsulation of substantially hydrophobic materials with a molecular weight greater than 700 Da (or 1000 Da) and/or a log P of at least 2 (or 3 or 4).

It should however be appreciated that materials of lower molecular weight (e.g. less than 700 Da) may be encapsulated, particularly for the case where such a material cannot be encapsulated using prior aqueous based processes. Thus for example the material may have a molecular weight of at least 100 Da, e.g. at least 400

Da.

The process is particularly effective for small to medium sized lipophilic molecules and macromolecules and molecules with partition coefficients above Log P 4.0 and a wider range of molecular weights from below 600 Da upwards and for poorly water soluble molecules. For example it is effective for aliphatic polysulfides with molecular weights ranging from less 1,000 to up to 5,000 Da.

It will also be useful for the encapsulation of halogenated compounds with logP above 4.0.

Depending on the nature of the microcapsule and the encapsulated material, the product may be for food, agrochemical, pharmaceutical or industrial use. Thus the encapsulated material can be a flavouring, essential oil, pharmaceutical, nutraceutical, or agrochemical. Thus method of the invention is effective for the encapsulation (for example) of insecticides (e.g. permethrin, deltramethrin, ivermectin, imidacloprid), fungicides (e.g. carboxin), molluscicides, (e.g. fentin & methiocarb), nematicides (e.g. carbofuran) rodenticides (e.g. brodifacoum, norbormide), herbicides (e.g. oxasulfuron) and poorly soluble active pharmaceutical ingredients (Class II and Class IV) (e.g. nifedipine, fenofibrate, griseofulvin, ketoconazole).

The polar aprotic solvent used in the method of the invention has a dielectric constant greater than 35 (measured at ambient temperature (25° C.). It is preferred that the polar, aprotic solvent has a dielectric constant of at least 40 and more preferably at least 45. Generally however preferred solvents will have dielectric constants less than 60. Solvents with a dielectric constant in the range of 45 to 50 are particularly suitable. The most preferred solvent for use in the invention is dimethyl sulfoxide (DMSO) which has a dielectric constant of about 47-48 at 25° C. Other solvents that may be used include N,N-dimethyl formamide (DMF) and dimethyl acetamide which have dielectric constants (at 25° C.) of 38 and 40 respectively. Generally however DMSO will be preferred, particularly for applications where solvent toxicity should be kept as low as possible.

The method of the invention is applicable to a wide range of microbial microcapsules such as algae, bacteria and fungi due to the presence of a protective polymeric envelope or cell wall. Most preferably the microcapsules are provided by fungal cells which may be derived from one or more fungi from the groups comprising Zygomycota, Glomeromycota, Ascomycota, Basidiomycota and Chytridiomycota. More preferably, the fungal cell is derived from yeasts. The most preferred fungi are Saccharomycetes, e.g. Saccharomyces cerevisiae, Saccharomyces boulardii, Torula yeast (Candida utilis) but may include Schizosaccharomycetes, e.g. Schizosaccharomyces pombe.

The microbial microcapsules may most conveniently be provided by bakers yeast, brewers yeast or yeast available as a bi-product of ethanol biofuel production (Saccharomyces cerevisiae).

The method of the invention may be effected with “live” microbial microcapsules but more preferably for convenience they are inactive or non-viable for ease of handling during processing.

As indicated above, the microbial microcapsules to be treated in step (a) of the process are preferably dry. Thus, for example, microbial microcapsules to be supplied to the process may initially be oven or spray dried or freeze dried. If the microcapsules are supplied with associated water (e.g. in the form of a slurry) then the capsules should preferably be separated from the free water as far as practicable.

The method of the invention will usually be carried out by admixture of the microbial microcapsules with a solution or dispersion in the polar aprotic solvent of the material that is to be encapsulated. It is in fact preferred that the material to be encapsulated is wholly soluble in the polar, aprotic solvent (at least at the level the material is present in the solvent). If however a dispersion of the material is used then at least a portion should be dissolved in the solvent. Generally therefore a solution or dispersion of the material to be encapsulated will be prepared and this will then be admixed with the microbial microcapsules. We do not however preclude the possibility that the microbial microcapsules are initially mixed with the solvent with the material to be encapsulated being added subsequently.

Generally the amount of material (to be encapsulated) in the polar organic solvent will be up to 50% by weight of the solvent although generally lower amounts will be employed, e.g. up to 25%, more preferably up to 10% and typically up to 5% (e.g. 1-5%) on the same basis.

Step (a) may be effected (usually with stirring or some other form of agitation) at ambient temperature, e.g. about 20° C. It is however generally preferred to operate step (a) at an elevated temperature but obviously not one which is so high that results in damage to the cell wall or cell membrane (since intact microbial cells will be required to retain the encapsulated material). The elevated temperature will serve to lower viscosity and result in better permeation of the material into the microbial microcapsule. Typically the elevated temperature can be up to 60° C. although more preferably will be in the range of 35-45° C. (and most preferably about 40° C.).

The procedure of step (a) will be continued until there is a desired level of incorporation of the material into the microcapsules. Typically this will involve effecting step (a) for a period of 1-16 hours although typically about 2 hours at a temperature of 40° C. will generally be found to be suitable.

At the end of step (a) the microbial microcapsules may be separated from the polar aprotic solvent and then subjected to a substantially aqueous environment for the purposes of step (b) of the process. Preferably the microcapsules from step (a) are initially treated with a liquid medium which comprises an admixture of the polar aprotic solvent (possibly in a major amount) and water and then, using successive aliquots of a mixture of the solvent and water containing increasing amounts of water relative to the polar aprotic solvent until the cells are washed with water (without polar aprotic solvent). This procedure reduces the degree of “shock” to which the microbial microcapsules are subjected.

After washing, the microbial microcapsules are preferably dried, e.g. by spray drying.

It is a significant feature of the method of the invention that, once encapsulated, the materials are not released by exposure of the microbial microcapsules to an aqueous environment, i.e. either in step (b) of the process or when water is added to dried microcapsules produced as outlined in the previous paragraph. Thus the retention properties of the microbial microcapsules are not effected by the treatment with the polar, aprotic solvent (e.g. DMSO) in step (a) of the process.

The invention is illustrated by the following non-limiting Examples and accompanying drawings, in which:

FIG. 1 shows bright field and fluorescence microscopy pictures of S. Cerevisiae exposed to a DMSO solution in accordance with the procedure of Example;

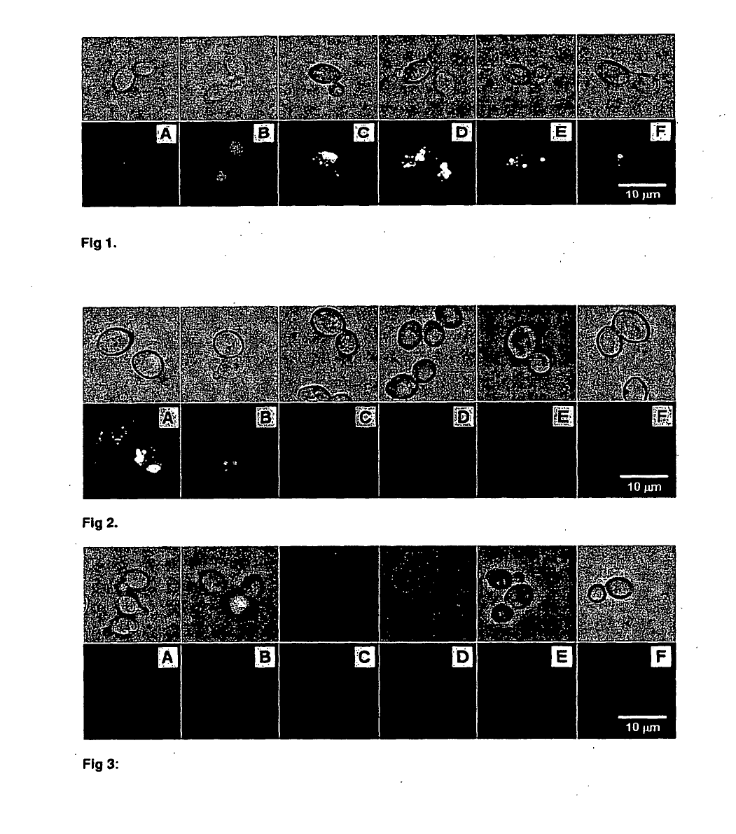

FIG. 2 shows bright field and fluorescence microscopy pictures of S. Cerevisiae exposed to 0.5 mM probe dispersions in water in accordance with the procedure of Comparative Example 1;

FIG. 3 shows bright and fluorescence microscopy pictures of S. Cerevisiae exposed to 0.5 mM probe dispersions in PBS after previous incubation for 2 h in DMSO in accordance with the procedure of Comparative Example 2; and

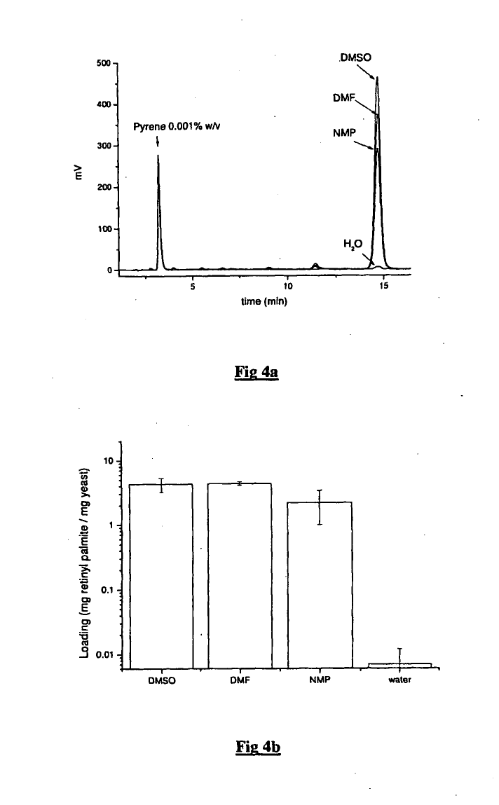

FIGS. 4a and 4b illustrate the results of Example 2.

### EXAMPLES

### 1. Preparation of Fluorescent Probes

Polysulfides with identical composition but different molecular weight were prepared. The preparative procedure was based on a one pot sequence of a) activation of a bifunctional initiator through deprotonation of 2,2′-(ethylenedioxy)diethanethiol by the means of a strong base (1,8-diazabicyclo [5.4.0]undec-7-ene, DBU), b) introduction of the monomers in variable monomer:thiol ratio to yield polysulfide chains with different molecular weights and terminal thiol groups and c) conjugation of fluorescent groups at the termini of the oligomer or polymer by using thiol-reactive fluorophores (Scheme 1). This procedure was used to produce probes designated as DA-1100, DA-1500, DA-2400 and DA-3800. These structures have close analogy to those present in garlic extracts that are known to be effectively encapsulated in yeast.

For the purpose of introduction fluorescent probes, we used dansyl acrylate which reacts through a Michael-type addition with terminal thiolates; we have used stoichiometric defects of this reaction to reduce the amount of molecules bearing two fluorophores, which, due to the limited molecular weight of the probes and thus the short distance between fluorophores, can produce emission quenching due to auto-absorption. The remaining thiolates were then end-capped with ethyl acetate groups to yield non-functional termini. Specifically, we have used dansyl acryate quantities corresponding to 25% of the amount of thiolates for most of the polysulfides (DA-1500 to DA-3800), or 10% for the shortest oligomer (DA-1100), where self-quenching could be more likely. We therefore produced probes that contained about 18.8% of single dansyl- and 6.2% of double dansyl-chains, or 2% and 8%, respectively (Table 1).

Low MW probes. Despite its living character, episulfide anionic polymerisation cannot produce narrow polydispersity samples at very low molecular weight. We therefore synthesised two low molecular weight fluorescent probes without a propylene sulfide backbone.

The lowest MW probe, Dansyl-hexylsulfonamide (DA-320), was obtained by direct reaction of hexyl amine with dansyl chloride and features a short aliphatic chain without sulphur atoms. We have used this probe as a model for a generic low MW, very hydrophobic substance (a blank).

A second probe was synthesised by direct reaction of the initiator of episulfide polymerisation with dansyl acrylate, to yield a “grade zero” fluorescently labelled sulfide. We have used the same two-step end-capping procedure used for polymers, separating the compounds with two, one or no dansyl groups through chromatographic elution (1:1 hexane:ethyl acetate on silica); for all further experiments we have used the compound featuring only one dansyl group (DA-620).

Probe characterisation. For the oligo- and polymeric probes, ¹H-NMR and GPC data are in good agreement to confirm the increasing MW with increasing theoretical degree of polymerisation (Table 1).

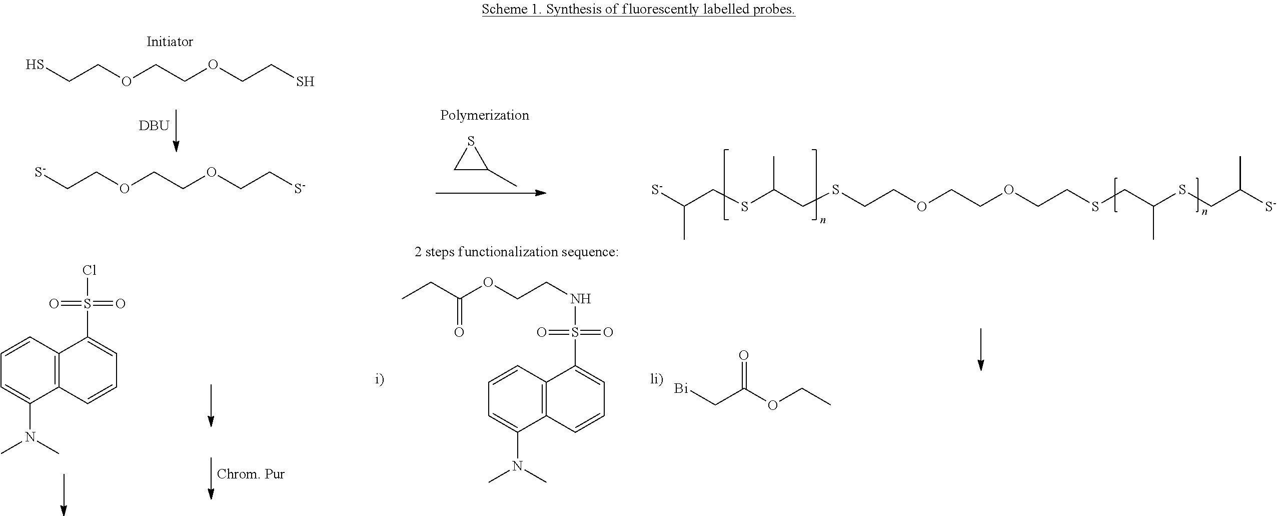

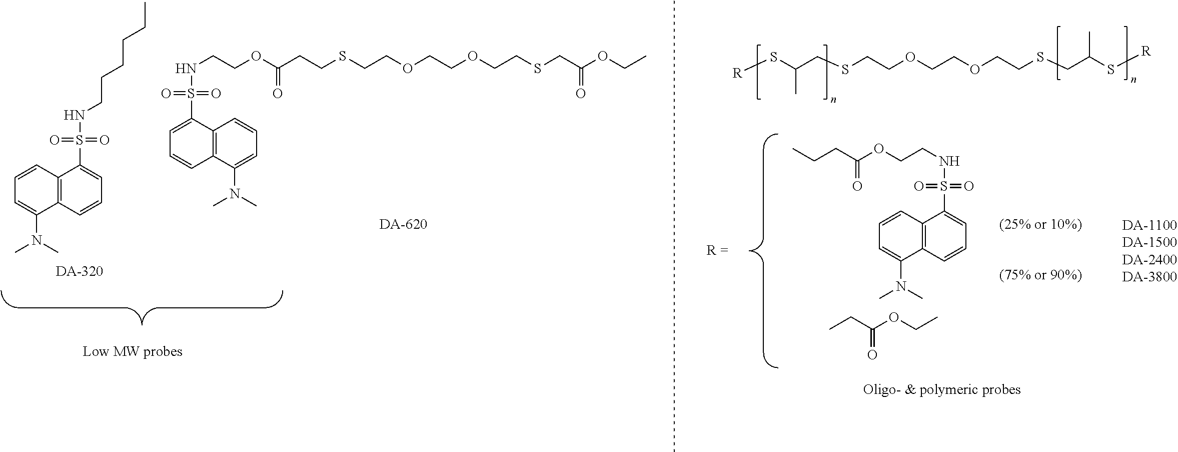

TABLE 1
Characterisation data for low MW and oligo/polymeric probes

| | | | | | | % of probes | |
|---|---|---|---|---|---|---|---|
| | | ¹H- | GPC | | Ex/ | with 2, 1 & | |
| | | NMR | | M̅_̅{̅w̅}̅ | Em ^{c} | no fluoro- | Yield |
| Probe | 2n ^{a} | M̅ₙ̅ ^{b} | M̅ₙ̅ | M̅ₙ̅ | (nm) | phore ^{d} | (%) |
| DA-320 | — | 320 | — ^{e} | — ^{e} | 337/492 | 0/100/0 | 0.53 |
| DA-620 | 0 | 620 | — ^{e} | — ^{e} | 343/494 | 0/100/0 | 0.55 |
| DA-1100 | 7 | 1060 | — ^{e} | — ^{e} | 343/494 | 2/8/90 | 0.60 |
| DA-1500 | 15 | 1580 | 1500 | 1.12 | 343/494 | 6.2/18.8/75 | 0.70 |
| DA-2400 | 30 | 2550 | 2350 | 1.18 | 343/494 | 6.2/18.8/75 | 0.75 |
| DA-3800 | 40 | 3680 | 3800 | 1.21 | 343/494 | 6.2/18.8/75 | 0.77 |

^{a} monomers per initiator in the feed.
^{b} For oligo/polymeric probes calculated as the ratio between the integral value of a PPS methyl proton (1.30 ppm) and that of a 2,2′-(Ethylenedioxy) diethanethiol proton (2.73-2.79 ppm). For low MW probes calculated from the molecular structure as confirmed from ¹H-NMR (and IR).
^{c} Excitation and emission maxima measured in CHCl₃.
^{d} Theoretical percentage of chains with double, single and no dansyl end-capping, calculated assuming a quantitative reaction of dansyl acrylate and an independent reactivity of the two polymer termini.
^{e} DA-1100 was out of range of our GPC columns (PLgel 5 μm MIXED-D, PLgel 5 μm MIXED-C, PLgel 10 μm MIXED-B).

### Methods, Part 2. Cell Preparation

Saccharomyces cerevisiae (wild type diploid BY4743) was routinely batch-grown under sterile condition in YPD (1% yeast extract, 2% bacteriological peptone and 2% glucose): 10⁶-10⁷ cells were inoculated in 50 ml growing medium and incubated at 29° C. (170 rpm) in an orbital shaking incubator (MODEL G25, New Brunswick Scientific CO. INC, Edison, N.J., USA). Cells were harvested at desired concentration, centrifuged at 3000 rpm for 3 min and after removal of the supernatant rinsed in deionised water. The suspension was then centrifuged and the pellets suspended at the desired concentration in the working medium: PBS buffer (Dulbecco A, PH 7.3), TE-Buffer (50 mM Tris-HCl, 0.15M NaCl, 5 mM EDTA, PH 7.5), TRIS buffer (10 mM Tris-HCl, PH 7.4), deionised water or organic solvents such as DMSO, DMF or NMP or DMSO.

“Methods, Part 3. Extraction of Hydrophobes from Loaded Yeast Cells and their Quantification Via HPLC

Encapsulation in water: 1 ml of a suspension containing 100 mg of yeast cells per ml water (sampled from the environment of encapsulation) was transferred in a 1.5 ml Eppendorf tube and centrifuged at 10.000 rpm for 3 min. After removal of the supernatant, the resulting pellets was re-suspended in 1 ml of water and transferred to a new tube. The operation was repeated three times.

Encapsulation in organic solvents: 1 ml of a suspension containing 100 mg of yeast cells per ml of organic solvent (sampled from the environment of encapsulation) was pelleted as described for water suspensions, then gradually transferred to an aqueous environment by repeated re-suspension/pelleting cycles in media with a progressively increasing water fraction (10, 25, 50, 75, and 100%).

The final water suspensions (both from encapsulation in water and in organic solvents) were centrifuged, and the pellets weighed and re-suspended in 200 μl of water added with 132 μl of zymolyase stock solution (approximately corresponding 2800 units) and incubated in an orbital incubator for 15-20 min at 37° C. at 600 rpm. 332 μl of CHCl₃/CH₃OH 60/40 were then added and the resulting suspension was incubated in an orbital incubator for 5 min at 37° C. at 600 rpm and then centrifuged at 10.000 rpm for 3 min; the lower organic phase was carefully removed with a 200 μl automatic pipette. This extraction procedure was repeated four times. The combined organic phases were then evaporated for 10 min at 40° C. in a rotatory evaporator, and dissolved in 400 μl of the solvent used as HPLC mobile phase added of a suitable volume of a stock solution of internal standard (e.g. 80 μl of 0.006% pyrene in the same solvent used as mobile phase for the HPLC elution for retinyl palmitate). The final solution was filtered with a 0.45 μm PVDF membrane filter before injection in the HPLC apparatus.

Using retinyl palmitate as a typical model hydrophobe, the HPLC analysis used a C18 column (Evolution RP-C18 column mounted on a system from Laserchrom HPLC Laboratories Ltd, Rochester, Kent England, featuring on a UV-vis diode array detector set at 325 nm) in isocratic mode with a 1:9 dichloromethane/methanol eluent mixture at 1 ml/min. Under these conditions, retinyl palmitate has an elution time of ca. 15 min with linearity in concentration proved between 0.015 and 1 mg/ml.

### Example 1

### Encapsulation of Large MW Hydrophobic Compounds (Invention)

Yeast cells, harvested and prepared as previously described, were suspended in DMSO and allowed for 10 minutes at 30° C. under gentle shaking to facilitate the diffusion of the organic solvent in the cell body. Cells were then centrifuged at 3000 rpm for 5 min and suspended in an equal volume of fresh DMSO yielding a final cellular concentration of 5*10⁷ cells/ml.

The fluorescent probes were dissolved in DMSO to prepare stock solutions of 20% wt solid content and were stored at −20° C. and protected from light prior to being used. 500 μl of cellular suspension in DMSO (5*10⁷ cells/ml) were centrifuged for 3 min at 5000 rpm and pellets were then separated from the solvent. The pellets were added of 50 μl of the probe solution in DMSO (20% wt) and incubated at 40° C. for 2 hours (1500 rpm), always protected from light. The tubes were centrifuged for 3 min at 5000 rpm to remove the supernatant. The resulting pellets were rinsed with DMSO and gradually transferred to an aqueous environment by progressively increasing the water fraction (0, 5, 10, 25, 50, 75 and 100%) after each rinsing cycle, which was Composed of centrifugation at 5000 rpm (3 min), complete removal of supernatant, re-suspension in fresh solution, transfer in new tube and shaking for 5 min (30° C., 1000 rpm).

Bright field and fluorescence microscopy pictures of the resultant yeast cells were then obtained and are shown in FIG. 1 (A: DA-320; B: DA-620; C: DA-1100, D: DA-1500, E: DA-2400, F: DA-3800 g/mol).

The results shown in FIG. 1 clearly demonstrate that all probes were efficiently encapsulated by the yeast cells and returned therein after the washing procedures. Thus use of the technique of the invention has allowed encapsulation of probes which are a molecular weight range of 32 (DA-320) to 2800 (DA-3800).

### Comparative Example 1

The fluorescent probes were dissolved in DMSO to prepare stock solutions that were 100, 50, 25, 12.5 and 2.5 mM in fluorescent groups. For the polymeric probes these concentrations were calculated using the theoretical numerical average molecular weight so that e.g. a 25 mM solution corresponds to: 9 mg/g (DA-320), 17 mg/g (DA-620), 146 mg/g (DA-1100), 87 mg/g (DA-1500), 140 mg/g (DA-2500), 202 mg/g (DA-3800). The solutions were then stored at −20° C. and protected from light prior to use.

Yeast cells were harvested in the late stationary phase and, following preparation as previously described, were finally suspended in PBS buffer (5.10⁷ cells/ml).

490 μl of suspension were transferred to a 1.5 ml centrifuge tube and 10 μl of probes solution in DMSO were added yielding a final concentration of the fluorescent dye of 2.0, 1.0, 0.5, 0.25 and 0.05 mM.

The dispersions were incubated in an orbital shaker (Eppendorf Thermomixer) at 40° C. for 30 minutes (1500 rpm) protected from light. Afterwards, cells were separated from the remaining hydrophobic phase repeating three times the following rinsing procedure: tubes were centrifuged at 3000 rpm for 3 min, the supernatant carefully completely removed; pellets were then suspended in fresh PBS and transferred each time in a new tube prior to repeat the cycle.

The resultant yeast cells were then investigated microscopically and it was noted that for all the probe concentrations tested only the low molecular weight probes (DA-320 and DA-620) became encapsulated in the yeast.

This result is demonstrated in FIG. 2 which shows bright field and fluorescence microscopy pictures for the yeast cells employing the probes at a concentration of 0.5 mM in water (A: DA-320; B: DA-620; C: DA-1100, D: DA-1500, E: DA-2400, F: DA-3800 g/mol).

### Comparative Example 2

Yeast cells were incubated and treated with DMSO in accordance with the procedure of Example 1 but in the absence of the fluorescent probes.

After the cells had been finally transferred to water they were subjected to the procedure described in Comparative Example 1 (i.e. treatment with the probes in dilute aqueous medium).

Microscopic investigation revealed that for all probe concentrations tested only the low molecular weight probes (D1-320 and DA-620) became encapsulated, i.e. the encapsulation properties of yeast in a water environment are unchanged due to DMSO treatment

This result is demonstrated in FIG. 3 which shows bright field and fluorescence microscopy pictures employed in the probes at a concentration of 0.5 mM in water (A: DA-320; B: DA-620; C: DA-1100, D: DA-1500, E: DA-2400, F: DA-3800 g/mol).

### Example 2

Ten millilitres of a yeast suspension with cells cultured in stationary phase were harvested by centrifugation at 4500 rpm for 5 min. The cell pellet was then re-suspended in 1 ml of a 9:1 water/organic solvent mixture, then centrifuged at 10,000 rpm for 3 min and re-suspended in mixtures with gradually increasing organic solvent fractions (25, 50, 75, and 100%). Cells were finally centrifuged at 10.000 rpm for 3 min, and the pellet (100-110 mg) was re-suspended in 1 ml of the same solvent where 100 mg of retinyl palmitate (FW=524.86 Da) was previously dissolved. The suspension was agitated in an orbital incubator at 600 rpm, 40° C. for 2 h, then centrifuged at 10.000 rpm for 3 min and analyzed as described under Methods, part 3.

The procedure of the previous paragraph was carried out separately with DMSO and DMF as organic solvents for use in accordance with the invention and also with N-methylpyrrolidone (NMP) as a comparative solvent (dielectric constant=32). It was also carried out with water only (i.e. no organic solvent). The results are shown in FIGS. 4a and 4b. FIG. 4a shows HPLC chromatograms of retinyl palmitate extracted from yeast after encapsulation with different solvents (eluent: CH₃OH:CH₂Cl₂ 90:10, at 1 ml/m). FIG. 4b shows loading of retinyl palmitate (in mg per mg of yeast) using different solvents for the encapsulation.

The results are summarised below.

A retinyl palmitate loading of 4.3% in weight (average over three experiments) was obtained using DMSO as a solvent in accordance with the inveniton, corresponding to an encapsulation efficiency of retinyl palmitate (weight of encapsulated active divided by the total weight of the active) of about 4%

A retinyl palmitate loading of 4.4% in weight (average over three experiments) was obtained using DMF as a solvent in accordance with the invention, corresponding to an encapsulation efficiency of retinyl palmitate (weight of encapsulated active divided by the total weight of the active) of about 4%

In comparison, a retinyl palmitate loading of only 2.2% in weight (average over three experiments) was obtained using NMP as a comparative solvent, corresponding to an encapsulation efficiency of retinyl palmitate (weight of encapsulated active divided by the total weight of the active) of about 2%

A retinyl palmitate loading of 0:006% in weight (average over three experiments) was obtained using water as a dispersant, corresponding to an encapsulation efficiency of retinyl palmitate (weight of encapsulated active divided by the total weight of the active) of less than 0.01%.

### SUMMARY

The above experimental results demonstrate the effectiveness of the present Invention for the encapsulation of compounds that, due to their molecular weight or chemical composition are not effectively encapsulated through the process in a water environment. It was noted that

a) the procedure of Example 1 (invention) resulted in encapsulation of fluorescent probes even with high molecular weight as a result of the treatment with DMSO, these probes remaining entrapped after subsequent treatment of the yeast cells with water. In contrast Comparative Example 1 demonstrated that only the low molecular weight probes (DA-320 and DA-620) were able to enter the yeast cells when the yeast was contacted with the probes in a water environment. Additionally Comparative Example 2 demonstrates that only the low molecular weight probes were able to enter the yeast cells even after they had been subjected to an initial incubation with dimethyl sulfoxide and then transferred to water, showing that the treatment with DMSO did not modify permanently the encapsulation possibilities of the yeast cells.

b) the procedure of Example 2 resulted in encapsulation of retinyl palmitate as a result of the treatment with DMSO, DMF, or, to a lesser extent, NMP, this compound being encapsulated in negligible amounts in a water environment (improvement of 500:1 by comparing the procedures in DMSO and in water).

Taken together, the results of Example 1 and Comparative Examples 1 and 2 demonstrate that the DMSO is able to induce, in the cell wall of the yeast, a change which allows material to be encapsulated to pass across the wall. However, this change is reversible in that subsequent treatment of the yeast cells with water ensures that the incorporated material remains entrapped. In this respect it will be appreciated that the results of Comparative Example 2 demonstrate that yeast cells treated with dimethyl sulfoxide and subsequently re-suspended in water do not allow the high molecular probes to pass to the interior of the cell.

The results of Example demonstrate, on the other hand, that other solvents characterized by high dielectric constant can be used too.

1. A method of encapsulation comprising the steps of
(a) treating microbial microcapsules with a material to be encapsulated in a substantially anhydrous liquid medium containing a polar aprotic solvent having a dielectric constant greater than 35 under conditions to permit encapsulation of the material, and
(b) subjecting the product of step (a) to a substantially aqueous liquid.

2. A method as claimed in claim 1 wherein the polar aprotic solvent has a dielectric constant of at least 40.

3. A method as claimed in claim 1 wherein the polar aprotic solvent has a dielectric constant of at least 45.

4. A method as claimed in claim 3 wherein the polar aprotic solvent has a dielectric constant in the range of 45 to 50.

5. A method as claimed in claim 4 wherein the polar aprotic solvent is dimethyl sulfoxide (DMSO).

6. A method as claimed in claim 1 wherein the material to be encapsulated is water insoluble.

7. A method as claimed in claim 1 wherein the material to be encapsulated has a log P value greater than 2.

8. A method as claimed in claim 7 wherein the material to be encapsulated has a log P value greater than 3.

9. A method as claimed in claim 8 wherein the material to be encapsulated has a log P value greater than 4.

10. A method as claimed in claim 1 wherein the material to be encapsulated has a molecular weight greater than 400 Da.

11. A method as claimed in claim 10 wherein the material to be encapsulated has a molecular weight greater than 700 Da.

12. A method as claimed in claim 11 wherein the material to be encapsulated has a molecular weight greater than 1000 Da.

13. A method as claimed in claim 12 wherein the material to be encapsulated has a molecular weight of at least 2000 Da.

14. A method as claimed in claim 1 wherein the material to be encapsulated has a log P value greater than 2 and a molecular weight greater than 700 Da.

15. A method as claimed in claim 14 wherein the material to be encapsulated has a log P value of at least 3 and/or a molecular weight of at least 1000 Da.

16. A method as claimed in claim 1 wherein the microbial microcapsules to be treated in step (a) are dried and the liquid medium is the polar, aprotic solvent in anhydrous form.

17. A method as claimed in claim 1 wherein step (b) involves treatment of the microbial microcapsules with solvent-free water in the absence of polar aprotic solvent.

18. A method as claimed in claim 17 wherein the microcapsules from step (a) are initially treated with a liquid medium which comprises an admixture of the polar aprotic solvent and water and then with successive aliquots of a mixture of the solvent and water containing increasing amounts of water relative to the polar aprotic solvent until the cells are washed with solvent-free water.

19. A method as claimed in claim 1 wherein the microbial microcapsules are provided by algae, bacteria or fungi.

20. A method as claimed in claim 19 wherein the microbial microcapsules are provided by a yeast.

21. A method as claimed in claim 20 wherein the yeast is Saccharomyces cerevisiae.

22. A method as claimed in claim 1 further comprising the step of drying the product of step (b).
